(19) United States
(12) Patent Application Publication
(10) Pub. No.: US 2019/0321008 A1
(43) Pub. Date: Oct. 24, 2019

(54) SIZE-SELECTIVE SAMPLER

(71) Applicant: National Taiwan University, Taipei (TW)

(72) Inventors: Sheng-Hsiu Huang, Taipei (TW); Ting-Ju Chen, Taipei (TW); Yu-Mei Kuo, Taipei (TW); Chih-Wei Lin, Taipei (TW); Chih-Chieh Chen, Taipei (TW)

(21) Appl. No.: 16/007,223

(22) Filed: Jun. 13, 2018

(30) Foreign Application Priority Data
Apr. 20, 2018 (TW) 107113552

(51) Int. Cl. A61B 10/00

(52) U.S. Cl. CPC A61B 10/00; A61B 2010/0087

(57) ABSTRACT

A size-selective sampler includes: a base body having a receiving space and a concave surface curved toward the receiving space, wherein the concave surface has opposite first and second sides; an inlet port formed on the first side of the concave surface and communicating with the receiving space; and an outlet port formed on the second side of the concave surface and communicating with the receiving space. The inlet port has a length of at least 20 mm along the first side of the concave surface so as to reduce penetration of particles in an air flow. Further, the particle penetration curve is roughly equal to the ISO curve.

### BACKGROUND

### 1. Technical Field

The present disclosure relates to size-selective samplers, and, more particularly, to a size-selective sampler that meets international standard penetration requirements.

### 2. Description of Related Art

Personal respirable samplers are often used to sample workers in industrial areas. Current aerosol size-selective sampling adopts the criteria specified by the American Conference of Governmental Industrial Hygienists (ACGIH), the International Standards Organization (ISO) and the Comite Europeen de Normalisation (CEN). The respirable aerosol particles are defined by the British Medical Research Council (BMRC) and ACGIH in 1985.

A conventional cyclone size-selective sampler such as a virtual cyclone sampler disclosed in Taiwan Patent No. 424, 570 generates eddy currents and collects particles through inertial impacts. When thrown onto a wall by centrifugal forces and coming into contact with the wall, the particles may deposit, bounce, slide and even roll, which depend on the characteristics of the particles and the wall. Therefore, the particle separation efficiency curve of the cyclone size-selective sampler may shift due to the particle load.

In the conventional cyclone size-selective sampler, the loading effect may be affected by a variety of factors, including the type of particles, particle size distribution, environmental temperature and humidity, the structure of the sampler and so on. Particles of different compositions may have different penetration curves under the loading effect. It is shown that when solid particles are used for a loading test, a significant number of particles accumulate on an inner wall of the sampler, causing particles that subsequently enter into the sampler to impact on the inner wall, bounce or collapse and thereby changing the predetermined size-selective efficiency of the size-selective sampler. It is also shown that compared with large particles, small particles are more prone to accumulate on the wall to form a dust hill. The reason is that the larger particles have strong inertial impact forces and when impacting on the inner wall, the larger particles push the accumulated particles downward, thus removing the accumulated particles, forming a clean area on the impact surface and alleviating the loading effect to some extent.

Environmental humidity may also affect the loading effect. In an environment with high humidity, water molecules are attached to particle surfaces and modify the characteristics of the particle surfaces. As such, compared with a dry environment, the particles in the environment with high humidity are more prone to be attached to the inside of the sampler and aggravate the loading effect.

Since different particles have different adhesive characteristics, they have different influences on the loading effect. However, since the solid particle load can increase the capture efficiency of the sampler (i.e., the penetration curve shifts towards the direction of small particles), the number of particles collectable by a filter paper at the rear end of the size-selective sampler is reduced, thus causing underestimation of the sampling result and increasing the probability of error in health hazard analysis and risk assessment.

Therefore, how to overcome the above-described drawbacks has become critical.

### SUMMARY

In view of the above-described drawbacks, the present disclosure provides a size-selective sampler, which comprises: a base body having a receiving space and a concave surface curved toward the receiving space, wherein the concave surface has opposite first and second sides; a first port formed on the first side of the concave surface and communicating with the receiving space, wherein the first port has a length of at least 20 mm along the first side of the concave surface; and a second port formed on the second side of the concave surface and communicating with the receiving space.

In an embodiment, the receiving space has a length equal to that of the first side or the second side of the concave surface.

In an embodiment, the length of the receiving space is at least 20 mm.

In an embodiment, the receiving space has a width of at least 10 mm.

In an embodiment, the receiving space has a height of at least 10 mm.

In an embodiment, the first port has a width of from 0.35 mm to 1.25 mm.

In an embodiment, the second port has a width of from 0.35 mm to 1.25 mm.

In an embodiment, the first port or the second port protrudes to a region outside of the receiving space.

In an embodiment, the size-selective sampler further comprises a filter portion integrally formed on the second port. In another embodiment, the filter portion has a channel communicating with the second port.

Conventionally, an air flow, when entering into a sampler, comes into contact with a substantially vertical contact surface (an inner wall or an impact plate), and hence particles may stay on the contact surface by impact forces, thus resulting in a loading effect. In contrast, the size-selective sampler according to the present disclosure provides a concave track (a first path) and allows particles to deviate from main air flow lines according to different inertial forces. As such, small particles may follow the air flow along the first path due to small inertial impact forces, and large particles may deviate from the first path due to large inertial impact forces. Therefore, the present disclosure effectively eliminates serious errors caused by the conventional loading effect.

Further, the present disclosure overcomes the drawbacks of slightly insufficient capture efficiency of the large particles and overestimation of the ISO curve due to the large particle penetration in the virtual cyclone sampler of TW 424,570. In particular, since the length of the first port along the first side of the concave surface is at least 20 mm, the air flow has a certain flow rate, and the particles keep moving at a certain speed. As such, the large particles follow a cyclone and stay in the receiving space due to the inertial forces. Therefore, the size-selective sampler according to the present disclosure increases the capture efficiency of the large particles, and the overall penetration curve of the size-selective sampler is roughly equal to the ISO curve. Accordingly, the present disclosure avoids overestimation of the sampling concentration at the beginning of sampling. Furthermore, during the sampling process, the efficiency curve of the size-selective sampler does not shift significantly due to the particle load. Therefore, the present disclosure overcomes the drawback of underestimation of the sampling result due to the particle load in the conventional cyclone size-selective sampler. Hence, the present disclosure reduces the probability of error in health hazard analysis and risk assessment.

### BRIEF DESCRIPTION OF DRAWINGS

FIG. 1A is a schematic perspective view of a size-selective sampler according to a first embodiment of the present disclosure;

FIG. 1B is a schematic cross-sectional view of FIG. 1A;

FIG. 2A is a schematic perspective view of a size-selective sampler according to a second embodiment of the present disclosure;

FIG. 2B is a schematic side perspective view of FIG. 2A;

FIG. 2C is a schematic partially perspective view of FIG. 2A;

FIGS. 3A and 3B are schematic side perspective views of a size-selective sampler according to a third embodiment of the present disclosure;

FIG. 4 is a schematic perspective view showing application of the size-selective sampler according to the present disclosure; and

FIG. 5 is a graph showing experiment results of the size-selective sampler according to the present disclosure and the conventional size-selective sampler.

### DETAILED DESCRIPTION OF EMBODIMENTS

The following illustrative embodiments are provided to illustrate the disclosure of the present disclosure, these and other advantages and effects can be apparent to those in the art after reading this specification.

It should be noted that all the drawings are not intended to limit the present disclosure. Various modifications and variations can be made without departing from the spirit of the present disclosure. Further, terms such as “first,” “second,” “length,” “width,” “a” etc. are merely for illustrative purposes and should not be construed to limit the scope of the present disclosure.

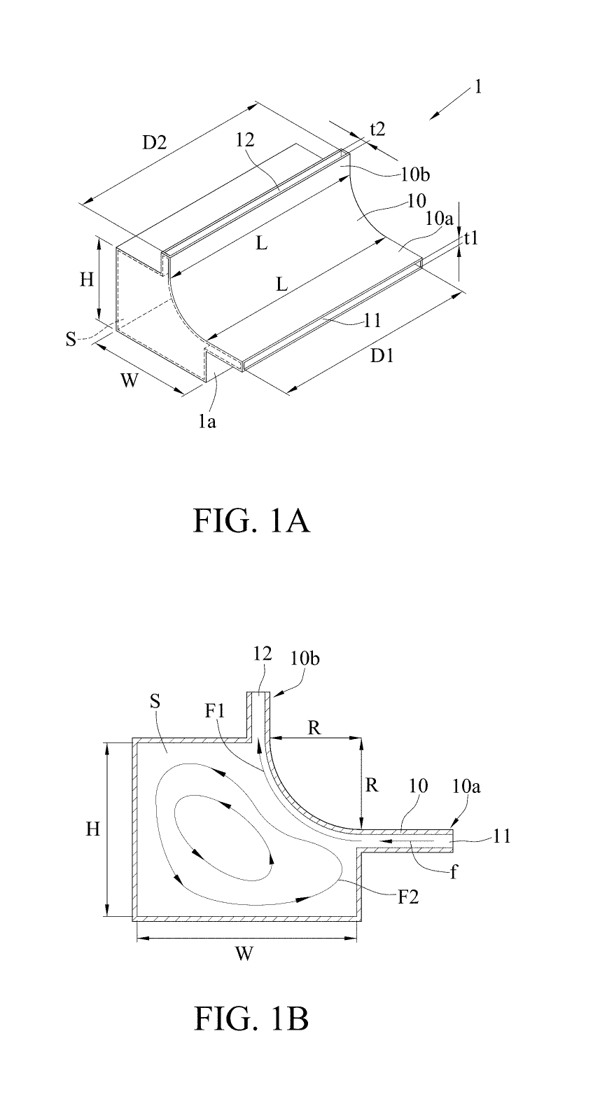

FIGS. 1A and 1B are schematic views of a size-selective sampler 1 according to a first embodiment of the present disclosure. The size-selective sampler 1 has a base body 1a, a first port 11 and a second port 12.

The base body 1a has a receiving space S and a concave surface 10 curved toward the receiving space S. The concave surface 10 has a first side 10a and a second side 10b opposite to the first side 10a.

In an embodiment, the base body 1a is formed from a rectangular hollow thin-walled body and the concave surface 10 is formed at an edge with a radius of curvature R (or by chamfering two adjacent planes). The receiving space S has a length approximately equal to the length L of the first side 10a or the second side 10b of the concave surface 10. In an embodiment, the length of the receiving space S is at least 20 mm Since the base body 1a is very thin in thickness, L≈20 mm.

In an embodiment, the receiving space has a height H of at least 10 mm and a width W of at least 10 mm.

In an embodiment, the first side 10a and the second side 10b of the concave surface 10 protrude above the outer surfaces of the hollow thin-walled rectangular body. In another embodiment, the first side 10a and the second side 10b of the concave surface 10 do not protrude above, but are flush with the outer surfaces of the hollow thin-walled rectangular body.

The first port 11 is formed on the first side 10a of the concave surface 10 and communicates with the receiving space S. The first port 11 has a length D1 of at least 20 mm along the first side 10a of the concave surface 10.

In an embodiment, the first port 11 is positioned at an interface between the concave surface 10 and one of the chamfered planes and protrudes above the receiving space S or the outer surface of the base body 1a. As such, the appearance of the size-selective sampler 1 corresponds to the receiving space S. In another embodiment, the length D1 of the first port 11 is equal to or less than the length L of the receiving space S. In yet another embodiment, the first port 11 does not protrude from the receiving space S, but is flush with the outer surface of the base body 1a and serves as an opening of the receiving space S.

In an embodiment, the first port 11 has a height t1 of 0.35 to 1.25 mm.

The second port 12 is formed on the second side 10b of the concave surface 10 and communicates with the receiving space S. In an embodiment, the second port 12 has a length D2 of at least 20 mm along the second side 10b of the concave surface 10.

In an embodiment, the second port 12 is located at an interface between the concave surface 10 and the other chamfered plane. The second port 12 protrudes above the receiving space S or the outer surface of the base body 1a. In an embodiment, the length D2 of the second port 12 is equal to or less than the length L of the receiving space S. In another embodiment, the second port 12 does not protrude from the receiving space S, but is flush with the outer surface of the base body 1a and serves as an opening of the receiving space S.

In an embodiment, the second port 12 has height t2 of 0.35 to 1.25 mm During operation of the size-selective sampler 1, the first port 11 serves as an air flow inlet port and the second port 12 serves as an air flow outlet port. When outside air containing particles with a diameter of 0.5 to 20 um enter into the receiving space S through the first port 11, a portion of the air flows along the concave surface 10 and leaves the receiving space S through the second port 12. Such a path is defined as a first air flow path F1 Small particles of the air can move along the first air flow path F1 and leave the receiving space S due to a low Strokes number. Another portion of the air forms a cyclone in the receiving space S. Such a path is defined as a second air flow path F2. Large particles of the air move along the second air flow path F2 (or along a tangential direction of the concave surface 10) and stay in the receiving space S due to an inertial force f.

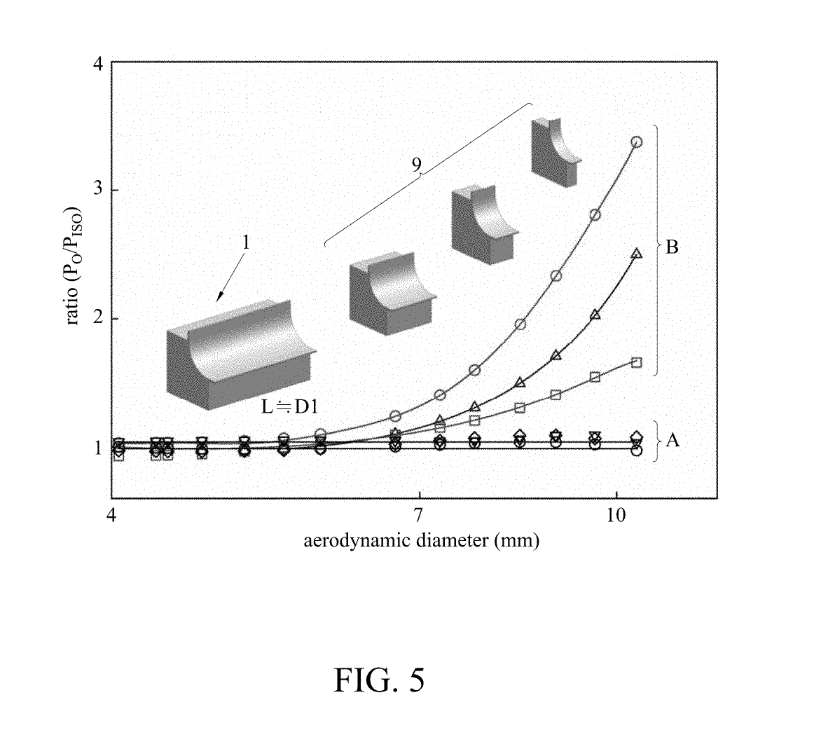

Therefore, since the length D1 of the first port 11 along the first side 10a of the concave surface 10 is at least 20 mm, the present disclosure greatly improves the capture efficiency of large particles. In an embodiment, if the length D1 of the first port 11 is less than 20 mm, when air enters into the size-selective sampler 1, a portion of the air will come into friction contact with the wall of the first side 10a, thereby decreasing the moving speed of particles and reducing the influence of the inertial force f on the particles. As such, large particles will move along the first air flow path F1 and leave the receiving space S. On the other hand, if the length D1 of the first port 11 is greater than or equal to 20 mm, the portion of the air coming into friction contact with the wall is reduced and therefore the large particles will move along the second air flow path F2 and stay in the receiving space S due to the inertial force f. As such, the length D1 of at least 20 mm facilitates to improve the capture efficiency of the large particles and hence the overall penetration curve of the size-selective sampler 1 is roughly equal to the ISO curve. Referring to FIG. 5, if the length D1 (D1≈L) of the first port 11 is at least 20 mm (for example, 20, 30, 40 mm), for particles of predetermined sizes, the ratios of the overall penetration curve P₀ to the ISO curve are roughly the same, as shown in straight lines A. If the length of the port of the conventional size-selective sampler 9 is less than 20 mm (for example, 5, 10, 15 mm), the ratios of the overall penetration curve P₀ to the ISO curve are not equal, as shown in curves B. Both the height H and width W of the receiving space S of the present disclosure are 20 mm, which are equal to the height and width of the receiving space of the conventional size-selective sampler 9, respectively.

In an embodiment, particle characteristics are measured by an aerodynamic particle spectrometer, such as an aerodynamic particle sizer (APS) Model 3321 from TSI Inc. The APS spectrometer uses a nozzle to produce an accelerated airflow and measures the time of flight (TOF) of a particle passing through two parallel laser beams so as to calculate the aerodynamic diameter of the particle. The size range measurable by the APS spectrometer is 0.5 to 20 um. Further, the APS spectrometer measures particle concentration and size distribution at the inlet port and the outlet port of the size-selective sampler so as to calculate the particle penetration curve. To observe the particle loading effect, the penetration experiment is performed over half an hour. Further, the APS spectrometer can provide monitoring data per second as a function of particle size. In addition to the size of the size-selective sampler, the effect of the sampling airflow on the load is also analyzed.

Since the length D1 of the first port 11 is required to be greater than or equal to 20 mm, the relationship between the height H and the width W of the receiving space S can be scaled to generate roughly the same penetration. In particular, the displacement of a particle on a relatively large radius of curvature R (having a relatively small radial velocity and the radial velocity being multiplied by the moving time) leaving the air flow track is equal to the displacement of a particle on a relatively small radius of curvature R leaving the air flow track. Therefore, regardless of the radius of curvature R, the size-selective sampler has the same size-selective efficiency.

If the length D1 of the first port 11 and the height H and width W of the receiving space S are determined (for example, 20 mm, 10 mm, 10 mm, respectively), the height t1 of the first port 11 or the height t2 of the second port 12 can be adjusted to provide a different air flow rate. If the height t1 of the first port 11 and the height t2 of the second port 12 are 0.35 to 1.25 mm, the size-selective sampler 1 has the same penetration, and the penetration curve is roughly equal to the ISO curve.

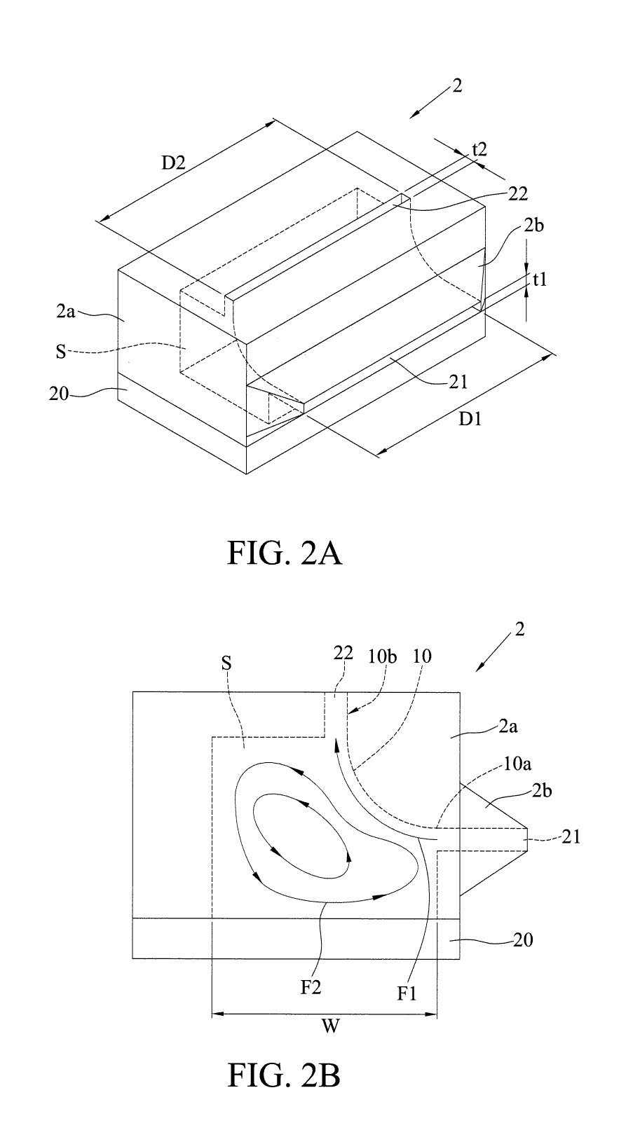

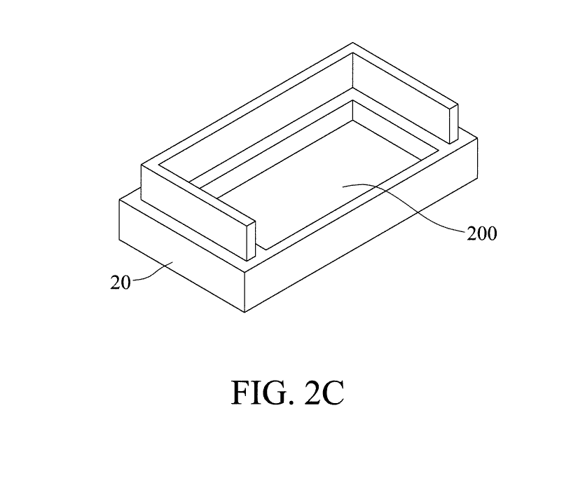

FIGS. 2A and 2B are schematic views showing a size-selective sampler 2 according to a second embodiment of the present disclosure. The second embodiment differs from the first embodiment in the appearance of the size-selective sampler.

Referring to FIGS. 2A and 2B, the base body 2a is substantially rectangular. A trapezoid protruding portion 2b is formed on one side of the base body 2a, and the first side 10a of the concave surface 10 extends to the protruding portion 2b. As such, the first port 21 is positioned on the protruding portion 2b, and the second port 22 extends from the receiving space S and is flush with the outer surface of the base body 2a.

In an embodiment, the base body 2a further has a carrier 20 disposed below the receiving space S to facilitate carrying of large particles in the air. In another embodiment, the carrier 20 of FIG. 2C has at least a recess 200 for collecting large particles.

Since the length D1 of the first port 21 of the size-selective sampler 2 is at least 20 mm, the present disclosure greatly improves the capture efficiency of large particles and allows the particle penetration curve to be roughly equal to the ISO curve.

Further, the length D1 of the first port 21 is required to be greater than or equal to 20 mm. As such, the relationship between the height H and width W of the receiving space S can be scaled to generate roughly the same penetration.

If parameters such as the length D1 of the first port 21 and the height H and width W of the receiving space S are determined and the height t1 of the first port 21 and the height t2 of the second port 22 are 0.35 to 1.25 mm, the size-selective sampler 2 has the same penetration and the penetration curve is approximately equal to the ISO curve.

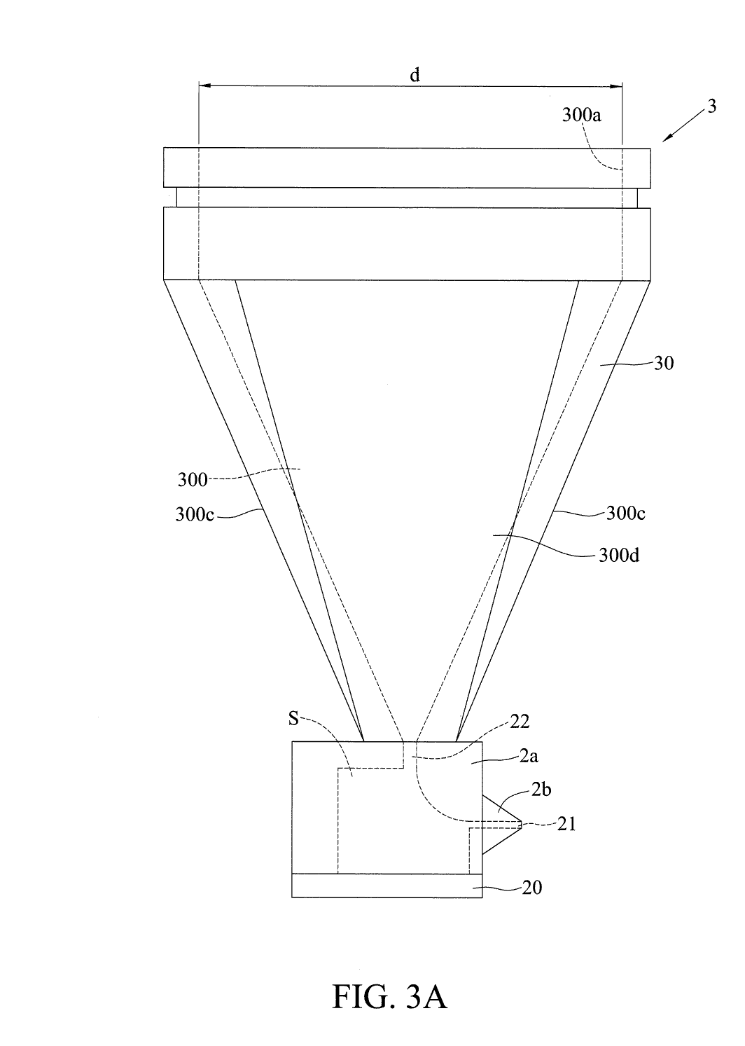

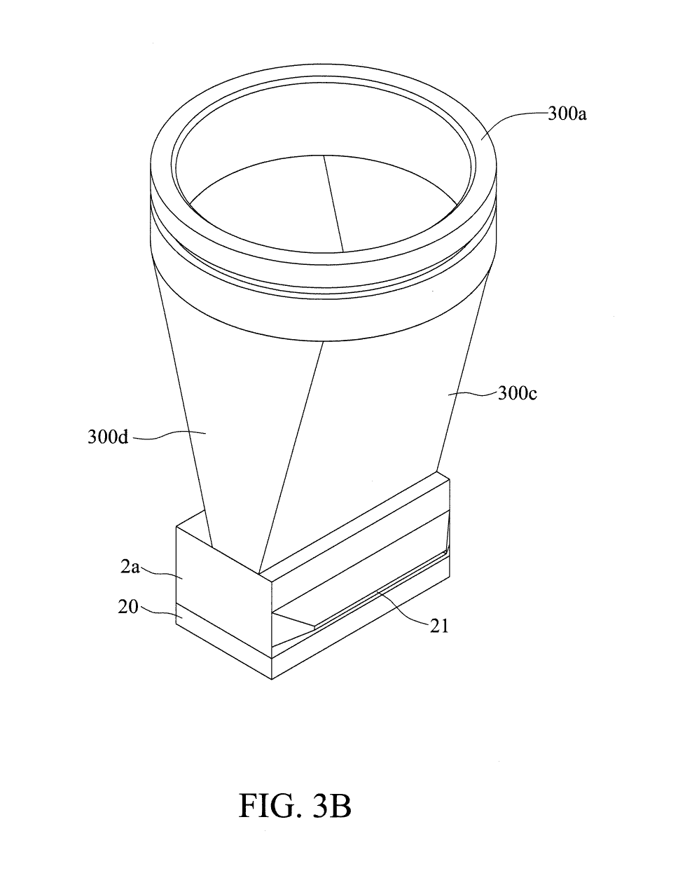

FIGS. 3A and 3B are schematic side perspective views of a size-selective sampler 3 according to a third embodiment of the present disclosure. The third embodiment differs from the second embodiment in the addition of a filter portion 30.

Referring to FIGS. 3A and 3B, the filter portion 30 is integrally formed on and extends from the second port 22. As such, a filter paper (not shown) can be disposed on the filter portion 30. In an embodiment, the filter portion 30 has a channel 300 communicating with the second port 22. A filter paper cassette (not shown) for storing the filter paper is disposed at an end 300a of the channel 300. In another embodiment, the width of the channel 300 gradually increases from the second port 22 toward the end 300a so as to form a tapered space. In yet another embodiment, two opposite sides 300c of the channel 300 along a direction of the first port 21 have flat surfaces (including inner and outer walls) and another two opposite sides 300d have curved surfaces (including inner and outer walls). These sides have the same width d at the end 300a and form a cylindrical shape to facilitate disposing of the filter paper cassette.

Since the length D1 of the first port 21 of the size-selective sampler 3 is at least 20 mm, the present disclosure greatly improves the capture efficiency of large particles and allows the particle penetration curve to be roughly equal to the ISO curve.

Further, the length D1 of the first port 21 is required to be greater than or equal to 20 mm. As such, the relationship between the height H and width W of the receiving space S can be scaled to generate roughly the same penetration.

If parameters such as the length D1 of the first port 21 and the height H and width W of the receiving space S are determined and the height t1 of the first port 21 and the height t2 of the second port 22 are 0.35 to 1.25 mm, the size-selective sampler 3 has the same penetration and the penetration curve is roughly equal to the ISO curve.

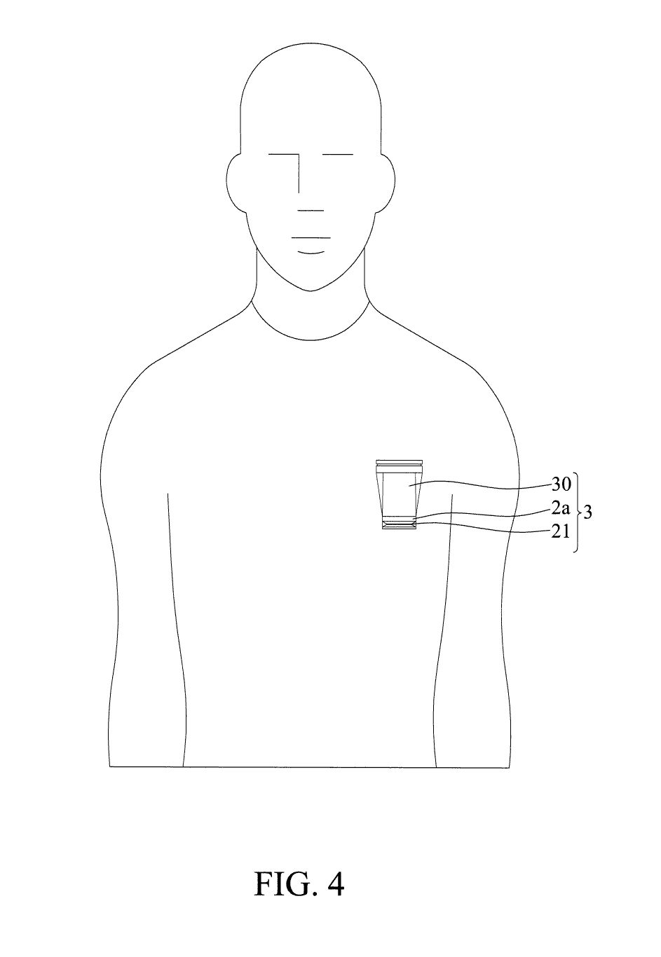

In an embodiment, the size-selective sampler 1, 2, 3 can be placed on a user's coat, as shown in FIG. 4. As such, environmental air flows into the size-selective sampler through the first port 21 and particles in the air are collected.

The above-described descriptions of the detailed embodiments are only to illustrate the preferred implementation according to the present disclosure, and it is not to limit the scope of the present disclosure. Accordingly, all modifications and variations completed by those with ordinary skill in the art should fall within the scope of present disclosure defined by the appended claims

What is claimed is:

1. A size-selective sampler, comprising:
a base body having a receiving space and a concave surface curved toward the receiving space, wherein the concave surface has opposite first and second sides;
a first port formed on the first side of the concave surface and communicating with the receiving space, wherein the first port has a length of at least 20 mm along the first side of the concave surface; and
a second port formed on the second side of the concave surface and communicating with the receiving space.

2. The size-selective sampler of claim 1, wherein the receiving space has a length equal to a length of the first side or the second side of the concave surface.

3. The size-selective sampler of claim 1, wherein the receiving space has a length of at least 20 mm.

4. The size-selective sampler of claim 1, wherein the receiving space has a width of at least 10 mm.

5. The size-selective sampler of claim 1, wherein the receiving space has a height of at least 10 mm.

6. The size-selective sampler of claim 1, wherein the first port has a width of from 0.35 mm to 1.25 mm.

7. The size-selective sampler of claim 1, wherein the second port has a width of 0.35 mm to 1.25 mm.

8. The size-selective sampler of claim 1, wherein the first port or the second port protrudes to a region out of the receiving space.

9. The size-selective sampler of claim 1, further comprising a filter portion integrally formed on the second port.

10. The size-selective sampler of claim 9, wherein the filter portion has a channel communicating with the second port.
